(19) United States
(12) Patent Application Publication
(10) Pub. No.: US 2004/0166533 A1
(43) Pub. Date: Aug. 26, 2004

(54) Optimization of synthetic catalysts by means of directed evolution

(76) Inventor: Manfred T. Reetz, Mulheim an der Ruhr (DE)

Correspondence Address: Norris McLaughlin & Marcus, 30th Floor, 220 East 42nd Street, New York, NY, 10017, US

(21) Appl. No.: 10/481,365

(22) PCT Filed: May 22, 2002
(86) PCT No.: PCT/EP02/05596

(30) Foreign Application Priority Data
Jun. 19, 2001 (DE) 10129187.6

(51) Int. Cl. G01N 33/53; C12N 9/00; G01N 33/543

(52) U.S. Cl. 435/7.1; 435/69.1; 435/183; 435/320.1; 435/325; 436/518

(57) ABSTRACT

The invention relates to a method for producing optimized catalysts which are optimized, during the course of said production method, in terms of a desired catalyst property or a plurality of properties. A synthetic metal-containing or metal-free catalyst is chemically linked to enzyme or protein mutants which are produced by means of molecular biology, and the catalysts created thereby are subjected to an optimization process using methods from molecular biology, or directed evolution of enzymes. The invention also relates to the catalysts which can be obtained by said method.

The present invention relates to the use of methods of directed evolution for optimizing the properties of metal-containing and metal-free catalysts by means of linking them to mutated enzymes or proteins. The invention furthermore relates to the catalysts which can be obtained thereby.

The importance of homogeneous catalysis in industrial practice is increasing constantly since, once they have been developed, efficient catalysts make it possible to achieve industrial conversions which are ecologically and economically attractive. The most important homogeneous catalysts include transition metal complexes. The ligands which are used in this connection assume two functions, namely that of stabilizing the metal and that of regulating the chemoselectivity, regioselectivity and stereoselectivity of the conversion which is to be catalyzed. The most common ligand types comprise compounds which contain one or more donor atoms for the metal complexation, for example nitrogen, oxygen, sulfur or phosphorus. To an increasing extent, transition metal-catalyzed processes are carried out in water or in two-phase systems (e.g. organic solvents/water), something which increases industrial attractiveness. Water-soluble ligands are required for this purpose. Whereas the stabilization of the metal by the currently known ligands can as a rule be regarded as being adequate, research during the last decade in the field of homogeneous transition metal catalysis has been concerned with the question of how industrially interesting selectivity and activity can be achieved by means of appropriate ligand tuning (Applied Homogeneous Catalysis with Organometallic Compounds, Eds.: B. Cornils, W. A. Hermann, Vol. 1+2, VCH, Weinheim 1996; Aqueous-Phase Organometallic Catalysis, Concepts and Application, Eds. B. Cornils, W. A. Hermann, VCH, Weinheim, 1998; Comrehensive Asymmetric Catalysis, Vol. I-III, Eds.: E. N. Jacobsen, A. Pfaltz, H. Yamamoto, Springer, Berlin 1999). Thus, the development of efficient chiral ligands for asymmetric catalytic conversion is, for example, regarded as being a particularly difficult challenge. While important advances have been made, for example as a result of the Sharpless asymmetric epoxidation of allyl alcohols, as a result of the same author's dihydroxylation of olefins, as a result of the Noyori hydrogenation of olefins and ketones, and as a result of the Jacobsen epoxidation of cis-olefins, these processes, like all the others, are in no way universally applicable. This means that, for a given industrially relevant conversion A→B, there may possibly not be a single chiral catalyst available which would catalyze this reaction with a high degree of activity and stereoselectivity.

Aside from the metal catalysts, some preparatively important metal-free catalyst systems exist in synthetic organic chemistry (F. A. Carey, R. J. Sundberg, Organische Chemie [Organic Chemistry], Eds.: H. J. Schäfer, D. Hoppe, G. Erker, VCH, Weinheim, 1995), e.g. bases or acids, NAD/NADH₂ analogs (NAD=nicotinic acid adenine dinucleotide) and thiazolium salts and also phase transfer catalysts. In this case, too, chiral versions have been developed in the case of stereorelevant conversions. A prominent example is that of the 4-N,N-dimethylaminopyridine-catalyzed acylation of alcohols in accordance with Steglich (G. Höfle, W. Steglich, H. Vorbrüggen, Angew. Chem. 1978, 90, 602). Chirally modified pyridines and phosphines catalyze the enantioselective acylation of chiral alcohols, as Fu and Vedejs have been able to demonstrate (J. C. Ruble, J. Tweddel, G. C. Fu, J. Org. Chem. 1998, 63, 2794; E. Vedejs, O. Daugulis, J. Am. Chem. Soc. 1999, 121, 5813). However, these and other metal-free catalyst systems are also subject to limitations, which means that, in this case, too, a given industrially relevant reaction A→B may in no way proceed with a sufficiently high degree of enantioselectivity. Similar restrictions apply to chemoselectivity and regioselectivity in relevant cases and to Z/E selectivity in olefin syntheses.

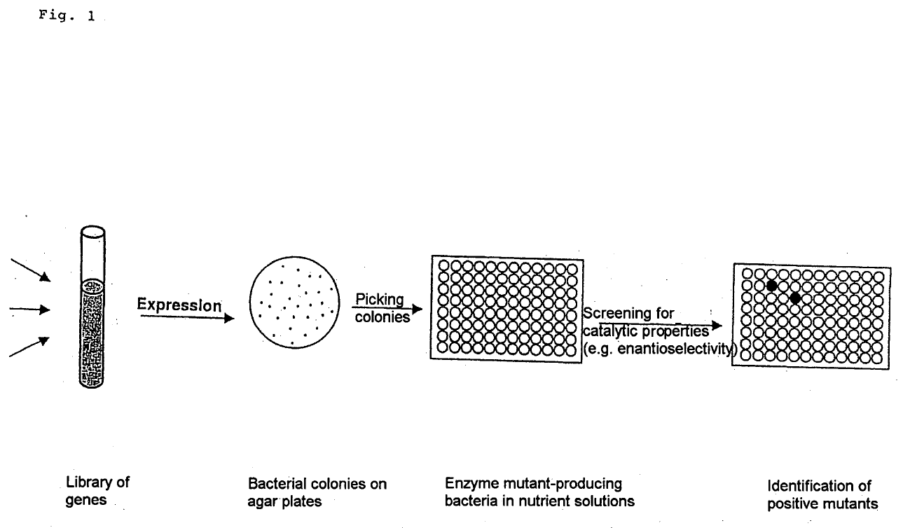

Biocatalysts, in particular enzymes, offer an alternative (A. Liese, K. Seebach, C. Wandrey, Industrial Biotransformations, WILEY-VCH, Weinheim, 2000). It has been discovered that a relatively large number of enzymes occurring in nature (wild-type enzymes) catalyze industrially relevant conversions. A perfect example of the enzyme-catalyzed industrial manufacture of a bulk chemical is that of the nitrile hydratase-catalyzed hydrolysis of acrylonitrile, resulting in the formation of acrylamide, in accordance with the Nitto process. Enzymes are also, and in particular, employed as chemoselective, regioselective and enantioselective catalysts in the field of fine chemical products. Limitations which are analogous to those applying to the abovementioned synthetic metal catalysts, and the metal-free catalyst, naturally also apply in this present instance, i.e. there is no certainty that, in the case of a given industrially relevant conversion A′→B′, that it can be automatically expected that the enzyme catalyst will exhibit a higher degree of selectivity. Problems of this nature can be solved using the new method of what is termed directed evolution such that new prospects for the industrial application of enzymes have very recently arisen (F. H. Arnold, Acc. Chem. Res. 1998, 31, 125; M. T. Reetz, K.-E. Jaeger, Top. Curr. Chem. 1999, 200, 31). The starting point is a conversion A′→B′ which an enzyme occurring in nature (wild-type enzyme) does catalyze but with an activity and/or selectivity which is too low. The corresponding gene (DNA segment) which encodes the enzyme is subjected to mutagenesis, with the formation of a library of mutated genes. After that, the mutated genes are inserted into suitable bacteria which then produce the encoded enzymes (expression system). This is done by the treated bacteria being plated out on agar plates, resulting in the formation of bacterial colonies, which are collected individually, added to the wells of microtiter plates and provided with nutrient solution. Each colony is derived from a single cell and therefore produces a single mutated enzyme. This results in the formation of thousands of differently mutated enzymes in which one or more amino acids in the protein chain have been replaced in a random manner. A suitable screening system is then used to test the enzyme mutants, which are frequently termed variants in the literature, for their catalytic properties in a given reaction A′→B′ (activity, chemoselectivity, regioselectivity and stereoselectivity) (see FIG. 1).

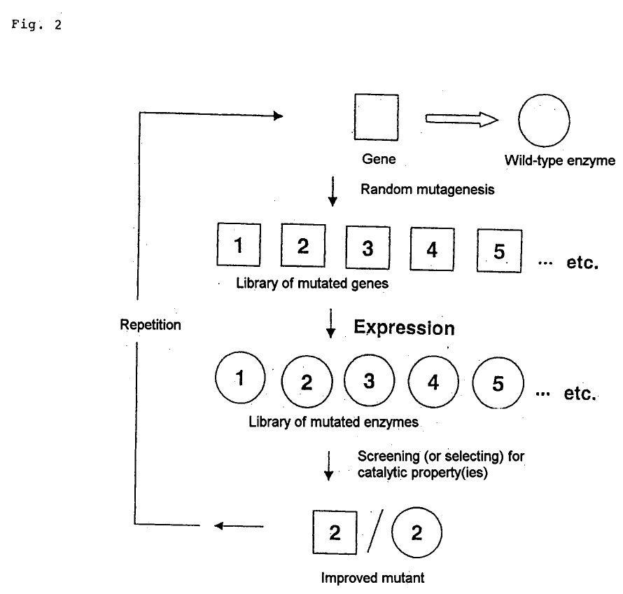

The best mutant (or one of the better mutants) is identified in the first generation of mutants and the corresponding mutated gene is once again subjected to mutagenesis, resulting in the development of an evolutionary pressure. The poor enzyme mutants, or those which are less active or selective, are screened out. Several cycles of mutagenesis/screening (selection) can be carried out until the desired catalytic property of the enzyme in the conversion A′→B′ has evolved. It is possible, in this way, to optimize activity, substrate acceptance and stereoselectivity. One of the great advantages of such a Darwinistic concept is that, in contrast to using site-specific mutagenesis to selectively replace amino acids, no theoretical predictions, which may possibly be insufficiently precise, are required in the case of this type of enzyme optimization. It is the evolutionary pressure alone which reliably and inevitably brings about the optimization (see FIG. 2). The directed evolution of enantioselective enzymes is of particular interest (M. T. Reetz, K.-E. Jaeger, Chem.-Eur. J. 2000, 6, 407).

The mutagenesis methods include the error-prone polymerase chain reaction (epPCR), saturation mutagenesis, cassette mutagenesis and, where appropriate, site-directed mutagenesis, and also recombinant methods such as DNA shuffling, combinatorial multiple cassette mutagenesis (CMCM) and the step method (F. H. Arnold, Acc. Chem. Res. 1998, 31, 125; M. T. Reetz, K.-E. Jaeger, Top. Curr. Chem. 1999, 200, 31). A combination of different methods is frequently advantageous for screening the expanse of the protein sequence in regard to a catalytic property. The rapid development of high-throughput screening systems (B. Jandeleit, et al., Angew. Chem. 1999, 111, 2648), for example in the field of enantioselectivity as well (M. T. Reetz, Angew. Chem. 2001, 113, 292), also plays a key role in using this approach. Despite the fact that the method of the directed evolution of enzymes constitutes such a reliable instrument, the whole approach suffers from one crucial disadvantage: the conversions are restricted to reactions which are catalyzed by enzymes. The very wide variety of industrially interesting reaction types which characterizes the field of homogeneous transition metal catalysis and the field of metal-free catalysis in synthetic organic chemistry is not found in enzyme catalysis and is thereby excluded. Thus, such important industrial processes as, for example, rhodium-catalyzed, ruthenium-catalyzed or iridium-catalyzed hydrogenations of olefins, rhodium-catalyzed hydroformylations of olefins, palladium-catalyzed C—C bond formations, nickel-catalyzed hydrocyanations of olefins, ruthenium-catalyzed olefin metatheses and palladium-catalyzed Ziegler-Natta polymerizations of olefins, to mention but a few, are not catalyzed by enzymes. Directed evolution cannot, therefore, be used to optimize these catalytic processes.

The present invention comprises a solution to this problem. This invention provides the practising chemist with a completely novel instrument which he or she can use to reliably optimize the catalytic properties of synthetic metal catalysts and/or metal-free catalysts. In addition, the catalysts, which are produced from enzyme mutants or protein mutants and metal-containing or metal-free synthetic catalysts, constitute a novel catalyst class.

The present invention consequently relates

(1) to a process for preparing a catalyst which is a synthetic metal-containing or metal-free catalyst which is linked to an enzyme or protein and which is optimized with regard to one or more desired catalytic properties, with the process comprising the usual methods of directed evolution to optimize the enzyme or the protein, linking a metal-containing or metal-free starting catalyst to the enzyme or protein, or modifying this catalyst with the enzyme or protein, and screening the resulting library of catalysts with a screening system which is suitable in regard to the catalytic properties;

(2) to catalysts which can be obtained, in accordance with (1), by chemically linking a synthetic metal-containing or metal-free catalytically active center to an enzyme mutant or protein mutant; and

(3) to the use of methods of directed evolution for identifying optimized enzymes or proteins which are suitable for being linked to metal-containing or metal-free catalysts.

The present invention is explained in more detail below with the aid of enclosed figures. In this regard

FIG. 1 shows a scheme for identifying specific mutants in a gene library,

FIG. 2 shows a scheme for using random mutagenesis to identify specific mutants of a starting gene,

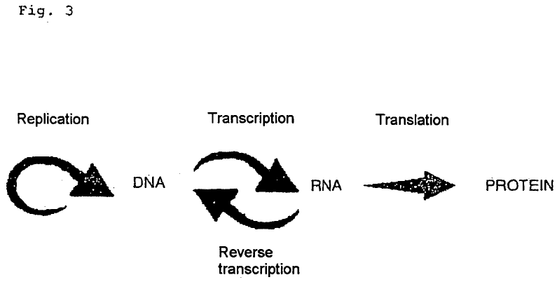

FIG. 3 shows a scheme indicating the flow of the genetic information,

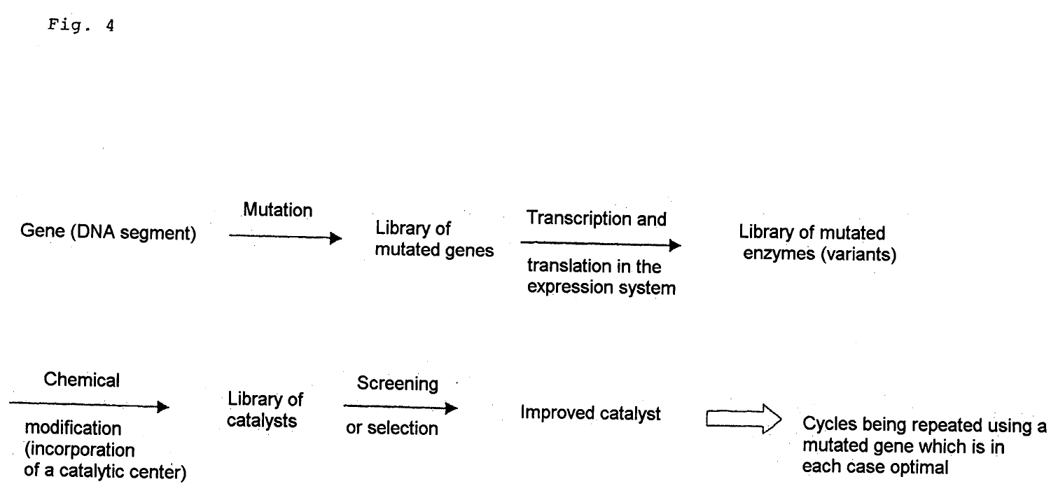

FIG. 4 shows a scheme of the process according to the invention for preparing optimized catalysts, and

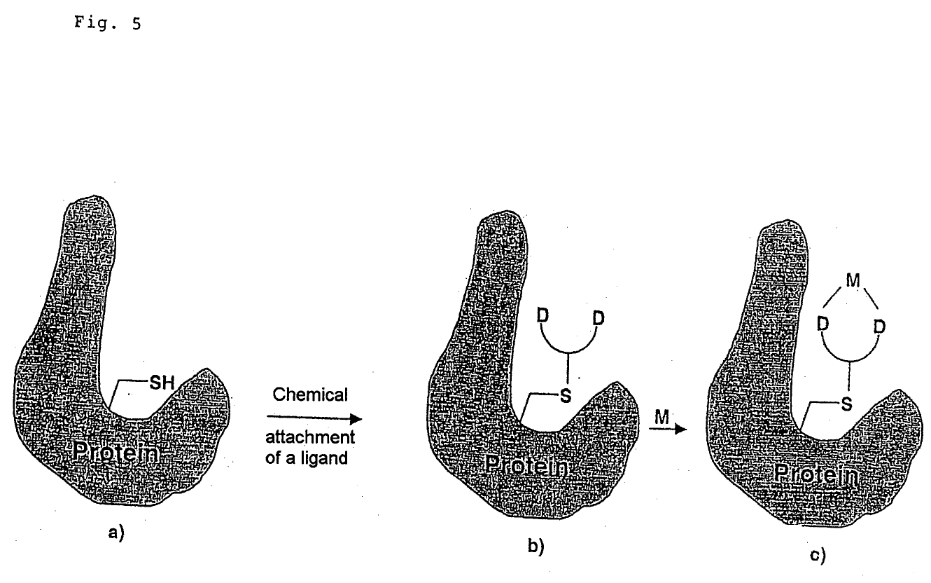

FIG. 5 shows a diagram of a catalyst in which a metal-containing catalytic center can be bonded to a ligand.

The starting point of the invention is the idea that the flow of genetic information, on which the concept of the directed evolution of enzymes is based, can be used in connection with optimizing a synthetic metal catalyst or a synthetic metal-free catalyst (see FIG. 3).

Embodiment (1) of the present invention consequently relates to a process [lacuna] of a catalyst which has been optimized, in regard to one or more desired catalytic properties, with enzymes or proteins, with methods of directed evolution and a screening system being used to select the suitable enzyme or protein from a library of enzymes or proteins, respectively.

In embodiment (1) of the present invention, “methods of the directed evolution of enzymes or proteins” means that the gene which encodes the wild-type enzyme or protein is subjected to mutagenesis. The flow of the genetic information of the individual mutated genes is effected in the expression system by a large number of mutated enzyme (or protein) mutants or variants being formed during the transcription to RNA and translation into the enzyme or protein. Within the meaning of the present invention, enzyme (or protein) “mutants” or “variants” differ from the starting enzyme or starting protein primarily by their amino acid sequence differing by at least one amino acid residue from that of the starting enzyme or starting protein. In addition to this, the mutants or variants can exhibit other chemical modifications (such as alkylation or acylation of the mutants).

While these enzyme mutants are normally tested for enzymic activity and selectivity within the context of a high-throughput screening system, what now initially takes place within the context of the invention is a chemical process which involves a synthetic metal-containing or metal-free starting catalyst (also termed a “metal-containing or metal-free center) being linked to the mutated enzymes or proteins. This thereby forms the actual “synthetic metal-containing or metal-free catalyst” within the meaning of the present invention (also term “hybrid catalyst” which, in contrast to the corresponding starting catalyst, is linked to one or more enzymes or proteins). If, for example, a specific ligand together with a defined metal is incorporated by means of covalently linking to the mutated enzymes (or proteins), this then results in the production of a large number of new catalysts which, while all containing the same catalytically active center, possess a different amino acid sequence in the enzyme or protein moiety of the overall catalyst. The members of such a library of catalysts therefore possess differing catalytic properties. The protein (or the “former” enzyme), into which the artificial catalytically active metal center, or the metal-free catalytic center, is chemically implanted, serves as a molecular host. The actual conversion to be investigated proceeds at the implanted metal-free catalytic center or at the metal which has been introduced. Since the amino acids of the enzyme or protein skeleton are able to sterically, electrostatically and chemically (e.g. by means of hydrogen bonds) influence the ligand system and the metal (or the metal-free catalytic center) as well as the reacting substrate, the enzyme or protein skeleton serves as an element for regulating the activity and selectivity of the overall catalyst. It can also serve for additionally stabilizing the catalytically active metal.

Within the meaning of embodiment (1) of the present invention, “screening” consequently denotes the scanning of a library of catalysts, which are linked to different enzyme or protein mutants, for at least one desired (i.e. to be optimized) catalytic property.

During the course of the first mutagenesis cycle, followed by the chemical insertion of the synthetic ligand/metal center or of the catalytically active metal-free center, a large number of new catalysts are formed, with these new catalysts possibly including one (or more) which is highly suitable for a given reaction A→B. If, as is conceivable, the activity and selectivity are not acceptable even in the case of the best catalyst, the mutated gene which encodes the corresponding mutated enzyme (or protein) is subjected to the mutagenesis once again. After a new generation of mutated enzymes (or proteins) has been expressed, the same artificial catalytically active center is once again chemically incorporated into all the enzyme (or protein) mutants, resulting in the formation of the second generation of catalysts. If these variants or mutants are once again screened as catalysts in the conversion A→B, this then produces an evolutionary pressure. Repeated cycles of mutagenesis/chemical modification/screening inevitably lead to a metal catalyst or metal-free catalyst being optimized for a given conversion A→B (FIG. 4). In this connection, it is possible to have recourse to the entire range of mutagenesis methods, such as epPCR, saturation mutagenesis, cassette mutagenesis, site-directed mutagenesis and recombinant methods such as DNA shuffling, or other mutagenesis methods which are normally used in connection with the directed evolution of enzymes. Any screening methods can be applied (B. Jandeleit, et al., Angew. Chem. 1999, 111, 2648), for example high-throughput assays for enantioselectivity (M. T. Reetz, Angew. Chem. 2001, 113, 292) as well in relevant cases. In addition to optimizing the activity of the catalyst, the method can also be used to optimize chemoselectivity, regioselectivity and stereoselectivity, for example. In the case of stereoselectivity, it is principally enantioselectivity and diastereoselectivity (including Z/E selectivity in the case of olefin syntheses) which come into consideration.

The process is consequently a process for preparing optimized catalysts which, during the course of the preparation process, are optimized in regard to a desired catalytic property or several properties, with a synthetic metal-containing or metal-free catalyst being chemically linked to enzyme or protein mutants produced by molecular biology and the resulting catalysts being subjected to an optimizing process using the methods of molecular biology or of the directed evolution of enzymes.

The choice of the enzyme or the protein is virtually arbitrary; in principle, any enzyme or protein can serve as the host for a synthetic, i.e. artificial, catalyst even if the protein itself does not possess any catalytic properties. However, within the context of the invention, there are practical guidelines which preferably have to be observed. A functioning expression system has to be available for the enzyme (or for the protein) since, otherwise, while it will be possible to mutagenize the gene or the genes, it will not be possible to produce the mutated enzymes (or proteins). Furthermore, the enzyme (or the protein) should preferably exhibit a thermal and chemical stability which is sufficiently high.

In a number of cases, it is useful to select an enzyme (or protein) which possesses a sufficiently large enzyme (or protein) pocket into which the artificial active center is placed or chemically bonded. This maximizes the interaction of the enzyme (or protein) skeleton with the artificial active center and/or with the substrate which is involved in the conversion. However, it is also possible to select other sites in the enzyme (or protein) for chemically docking the synthetic catalyst, for example on the surface of the enzyme (or protein), since, during the course of the evolutionary process, the folding of the enzyme (or protein) can quite possibly alter and consequently exert a greater influence on the catalytic properties of the overall catalyst.

During the procedure according to the invention, preferably only one synthetic, catalytically active center is incorporated into the enzyme (or protein) mutants, with this incorporation taking place by way of ionic bonds, but preferably by way of covalent bonds. For this, a reactive site in the enzyme (or protein), which site can, but does not have to, be the catalytically active center of the enzyme is reacted with a suitable reactive compound which contains the desired ligand system or catalytically active center, resulting the formation of a stable covalent bond. The formation of covalent bonds between active sites in enzymes with reactive organic compounds is known in principle. Thus, semisynthetic enzymes have been prepared, for example, by using substitution reactions or Michael additions to incorporate foreign cofactors at the cysteine thiol function in an enzyme (E. T. Kaiser, Angew. Chem. 1988, 100, 945).

In the present invention, a cysteine unit in the enzyme (or protein) can be used in a chemically similar manner. If the enzyme (or protein) does not contain any cysteine, it is possible to use site-directed mutagenesis to insert this amino acid at an arbitrary position in the enzyme or protein. If the enzyme (or protein) contains more than one cysteine in the amino acid chain, it is possible, where appropriate, to use site-directed mutagenesis to initially replace the unwanted cysteine units with any other natural amino acid. Whichever route is taken (preference is given to using an enzyme or protein which contains only one cysteine unit), the process according to the invention can, when cysteine is the reactive site and a catalytic center containing a metal is used, be depicted schematically as a bidentate ligand system which, proceeding from mutants a, each of which contains a thiol function, leads, by way of the chemically modified enzyme (or protein) mutants b possessing donor sites D, to metal complexes c containing the metal M (see FIG. 5).

The procedure according to the invention may be illustrated using this example. The gene for a wild-type enzyme (or protein), which, for example, only contains one cysteine unit, is subjected to the mutagenesis, the library of mutated genes (in the special case only one gene mutated by site-directing mutagenesis) is expressed in a suitable expression system, and the mutated enzymes (or proteins) are chemically modified and provided with metals in order, then, to be tested as catalysts in a conversion A→B using a suitable screening system. Repeated cycles of mutagenesis using the gene underlying the enzyme (or protein) which is in each case the best are passed through until the desired catalytic property has evolved. All the steps can be carried out on a small scale on microtiter plates using commercially available robot stations. The procedure is analogous in the case of a metal-free catalyst.

An example of one of many possibilities when selecting the enzyme (or the protein) is the known and commercially available papain (EC number 4.3.22.2); it is chemically stable (aqueous solutions are stable even at 90° C.) and contains a single cysteine unit, with this cysteine being present in a spatially relatively capacious enzyme pocket which is large enough to accept different ligands.

However, papain is only used for illustrative purposes and does not restrict the procedure according to the invention in any way. Other enzymes, for example what are termed the thermophilic enzymes as well, and also proteins without catalytic properties, can be used as the molecular host (“housing”) for the metal-containing or metal-free catalytic centers.

The invention is in no way restricted to using metal-containing catalytically active centers; it is also possible to incorporate metal-free catalysts (resulting in the metal complexation being dispensed with). Examples are basic centers (such as pyridines, phosphines or tertiary amines) for base catalysis and acid centers (e.g. sulfonic acids) for acid catalysis, thiazolium cations for catalyzing acyloin reactions, flavin units for catalyzing oxidation reactions and NAD/NADH centers for catalytic redox processes, to mention but a few examples.

The two most important routes for linking thiol functions to ligand-carrying (or catalyst-carrying) organic molecules make use of substitution reactions (alkylation) a) and Michael additions b), with X being a halogen (Cl, Br or I) or a tosyl unit (OTs) and A being an activating functional group (e.g. carbonyl function).

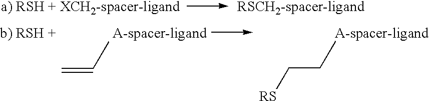

These are, therefore, reaction types which are common in organic synthetic chemistry. In the case of a), use is made of an S_{N}2-active alkyl halide (or tosylate). A spacer can, as desired, be located between the reactive XCH₂ function and the ligand. It is also possible to use other types of reaction for introducing catalytically active centers or ligands, for example customary addition/elimination reactions performed on aromatic moieties.

According to the invention, a very wide variety of ligands are suitable in the case of the metal-containing catalysts, provided these ligands are able to complex or stabilize metal. The skilled person in the field of organometallic chemistry, in particular metal catalysis, knows that both neutral and anionic ligands are used (J. P. Collman, L. S. Hegedus, J. R. Norton, R. G. Finke, Principles and Applications of Organotransition Metal Chemistry, University Science Books, Mill Valley, 1987). The anionic ligands include, for example, carboxylate anions, thiolates, amides, semicorrins, phosphonates and cyclopentadienyl anions. The most important neutral ligands are phosphorus-containing compounds such as phosphines, phosphites, phosphonites and phosphinites, nitrogen-containing compounds, such as nitriles, pyridines, amines, ketimines and oxazolines, and also compounds containing oxygen or sulfur as donor atoms (e.g. ethers, esters and thioethers). Those which are most well known are monodentate ligands possessing one donor site and bidentate or multidentate ligands possessing two or more donor atoms. Thus, diphosphines, dipyridines, dioxazolines, diketimines and diamines are examples of the most commonly used bidentate ligands. In addition to this, many mixed bidentate ligands, which possess two different donor sites, are known. In a number of cases, several metals are present in a ligand/metal system, for example as in the case of iron- or manganese-sulfur clusters. These latter can also be used in accordance with the invention.

Thus, any ligand types are suitable when functionalizing cysteine-containing enzymes (or proteins) at the thiol function provided these ligands are linked to a reactive function, such as an alkyl halide unit or a Michael acceptor, which enables a linking reaction to take place. The same applies to the insertion of metal-free catalytic centers. In every case, preference is given to inserting achiral ligands; however, their chiral analogs can also be inserted, if desired. Compounds 1-3, which react, during a substitution reaction, with the thiol function of cysteine-containing enzymes (or proteins), may be cited for illustrative purposes.

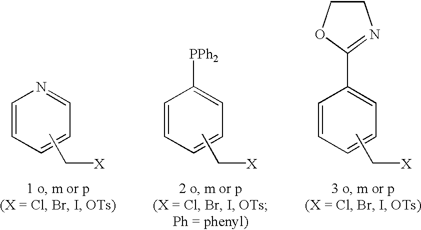

Cysteine-containing enzyme (or protein) mutants which have reacted with these or related alkylating agents would, for example, be metal-free catalysts for alcohol acylation reactions or Michael additions, since pyridines, phosphines and amines are known nucleophilic catalysts in such reactions (see above). It is naturally also possible, after the enzyme (or protein) mutants have been chemically modified, for metals to be complexed at the pyridine, phosphine or oxazoline centers in order, in this way, to generate metal catalysts which catalyze a large number of reactions. Acyloin reactions or oxidations and reductions can be catalyzed by enzyme (or protein) mutants which possess synthetically incorporated thiazolium functions, flavin functions or NAD/NADH₂ units. These examples only serve for illustrative purposes and in no way restrict the procedure according to the invention.

Compounds 4-8, which are likewise able to be covalently bonded to cysteine-containing enzyme (or protein) mutants by means of a simple S_{N}² reaction, may be cited for the purpose of illustrating bidentate ligands. In this case, too, metal-free and metal-containing catalysts can be obtained in an appropriate manner.

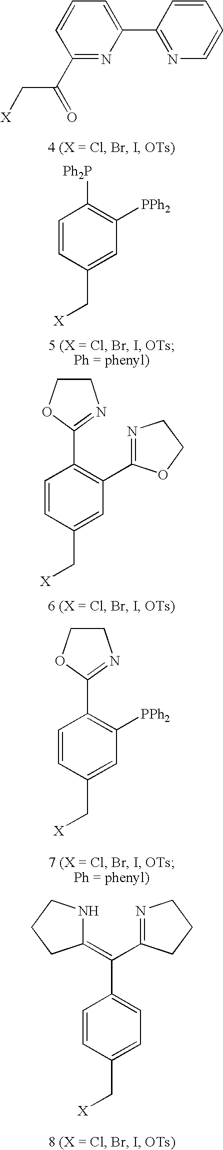

These, and other, ligand types can also be inserted into cysteine-containing enzyme (or protein) mutants using other methods of synthesis, for example during the course of a Michael addition. This requires corresponding, reactive Michael acceptors which, for their part, can be obtained using customary methods of synthesis. Compounds 9-14, which enter into straight-forward Michael additions with thiol functions, may be cited for illustrative purposes.

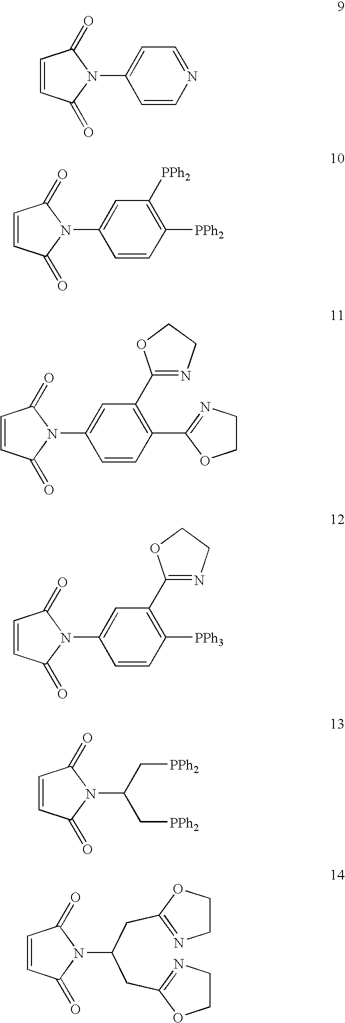

Compounds 1-14 only serve for illustrative purposes and in no way restrict the possibilities according to the invention. Thus, it is also possible, for example, to incorporate entire ligand/metal complexes directly into enzyme (or protein) mutants in one step as, for example, in the case of covalently bonding an iron complex to a cysteine-containing enzyme or protein. This is a known reaction type (J. M. Mazzarelli, et al., Biochemistry 1993, 32, 2779).

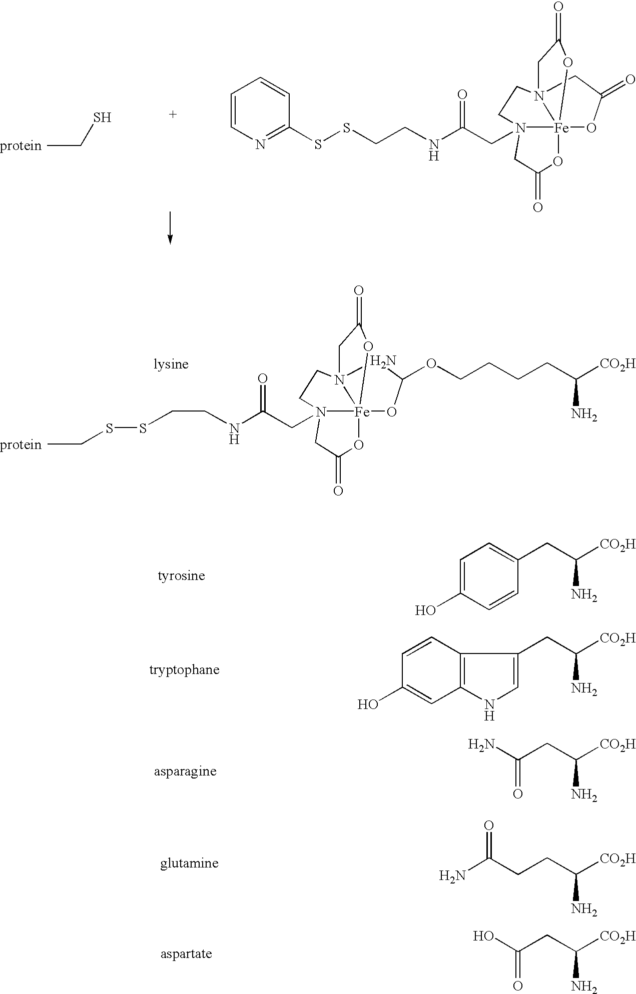

In this case, too, the example serves merely as an illustration and does not restrict the possibilities of the invention in any way.

Nor is cysteine the only amino acid which has a reactive functional group in the side chain. Lysine, tyrosine, tryptophan, asparagine, glutamine and aspartate provide further opportunities for inserting catalytically active centers into enzymes (or proteins). The same applies to cystine.

The proficient skilled person is familiar with many possibilities for linking the respective reactive side chains of these amino acids to ligands or to catalytically active centers. Thus, the primary amino group in the lysine side chain can, for example, be used in the following way, with the phosphinomethylation of primary amino groups being carried out as a known reaction type (M. T. Reetz, G. Lohmer, R. Schwickardi, Angew. Chem. 1997, 109, 1559):

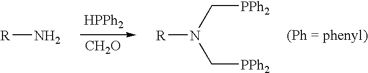

It is also possible to dispense with inserting ligands and to use the functional groups in the side chains of the amino acids in the mutated enzymes or proteins, for example in the case of cysteine, asparagine, glutamine or aspartate, directly for the purpose of metal coordination, with the functional groups in side chains of the amino acids preferably acting as anionic ligands (e.g. in order to coordinate rhodium or iron).

Basic functions such as pyridines, amines, phosphines and amino alcohols, come into consideration in the case of the metal-free systems since, as is known, these functions catalyze reactions such as the acylation of alcohols, Michael addition, cyanohydrin-forming reactions of aldehydes and Baylis-Hillman reactions. However, acids are also known to be metal-free catalysts; it is therefore also useful to selectively insert such functional groups into enzyme (or protein) mutants, e.g. in the form of sulfonic acid functions or phosphonic acid functions, e.g.:

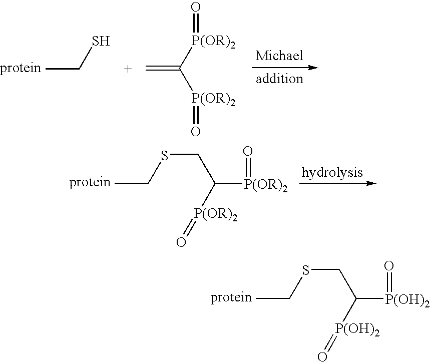

Important metal-free systems also comprise the insertion of thiazolium cations, flavin units and/or NAD/NADH₂ units, and also related redox systems, since it is then possible to catalyze acyloin reactions as well as oxidation processes and reduction processes.

As far as the metal-containing systems are concerned, it is, in particular, the transition metals of groups IIIb, IVb, Vb, VIb, VIIb, VIII, Ib and IIb which are suitable, as are boron, aluminum, indium and tin, as well as lanthanides, such as cerium or gadolinium, and also actinides, such as thorium.

The invention relates to metal-free and metal-containing catalytic processes in which a very wide variety of reaction types come into consideration. These include reductions and oxidations and, in addition, C—C bond formations and also addition reactions, isomerization reactions and substitution reactions. The catalytic properties which the invention can be used to optimize include activity, stability, chemoselectivity, regioselectivity, enantioselectivity and diastereoselectivity. The reactions can be carried out, for example, in water, in organic solvents, in two-phase systems (e.g. organic solvent/water, with the optimized catalyst also acting as a phase-transfer catalyst), in supercritical CO₂ or in ionic solvents. The catalysts are homogeneous or heterogeneous depending on solubility properties.

The catalysts which are optimized within the context of the invention can be used, inter alia, in the industrial production of bulk chemicals and of achiral and chiral fine chemicals, for example in the production of active compounds in the fields of pharmaceuticals, of perfumes or of phytoprotection. They can also be used in the production of dyes, of components in microelectronics and of polymers. They can also be used in the catalytic decomposition of toxic or environmentally harmful chemical compounds.

1. A process for preparing a catalyst, which is a synthetic metal-containing or metal-free catalyst which is linked to an enzyme or protein and which is optimized in regard to one or more desired catalytic properties, wherein the process [lacuna] using methods of directed evolution to optimize the enzyme or the protein, linking a metal-containing or metal-free starting catalyst to the enzyme or protein, or modifying the catalyst with the enzyme or protein, and screening the resulting library of catalysts with a screening system which is appropriate in regard to the catalytic properties, and wherein, proceeding from a gene which encodes a wild-type enzyme or protein,
(1) a library of mutated genes is generated by mutagenesis and from this,
(2) a library of enzymes or proteins is generated by way of an expression system, and
(3) the library of catalysts is generated by chemically linking the enzymes or the proteins to the metal-containing or metal-free synthetic starting catalyst, and
(4) a screening system is used to select a catalyst which is appropriate in regard to the desired catalytic property.

2. The process as claimed in claim 1, wherein
(5) the steps (1) to (4) or (5) are run through once again, proceeding from the mutated gene which corresponds to the enzyme or protein which was selected in the preceding step (4).

3. The process as claimed in claim 2, wherein the cycle described in steps (1-5) is run through as often as is required for the desired optimization of the catalytic property to be achieved.

4. The process as claimed in one or more of claims 1-3, wherein the enzyme mutants or protein mutants are generated by means of error-prone polymerase chain reaction (epPCR), saturation mutagenesis, cassette mutagenesis or site-directed mutagenesis or by means of recombinant methods or by means of combining these methods.

5. The process as claimed in claim 4, wherein DNA shuffling or combinatorial multiple cassette mutagenesis (CMCM) are used as recombinant methods for generating enzyme mutants or protein mutants.

6. The process as claimed in one or more of claims 1-3, wherein the modification of the synthetic metal-containing starting catalyst with the enzyme or protein mutants, and/or the linking of the catalyst to the enzyme or protein mutants, is effected by forming a covalent bond either directly with the metal, by means of a coordination, or by forming a covalent bond with a ligand system metal-containing or metal-free starting catalyst, followed by coordination with the metal, or by covalent bonding with a metal-containing ligand system.

7. The process as claimed in one or more of claims 1-6, wherein the modification of the synthetic metal-free starting catalyst with the enzyme or protein mutants, and/or the linking of the catalyst to the enzyme or protein mutants, is effected by means of forming a covalent bond.

8. The process as claimed in claim 7, wherein the formation of the covalent bond takes place at a functional group in the side chain of an amino acid which is located in the enzyme mutants or protein mutants.

9. The process as claimed in claim 8, wherein the amino acid is cysteine, cystine, lysine, tyrosine, tryptophan, asparagine, glutamine or aspartate.

10. The process as claimed in claim 9, wherein cysteine enters into either a nucleophilic substitution or a Michael addition with a synthetic ligand or a synthetic metal-containing or metal-free catalyst, resulting in the formation of appropriately modified enzyme or protein mutants.

11. The process as claimed in one or more of claims 1-3, wherein a high-throughput screening system is used.

12. The process as claimed in claim 11, wherein the high-throughput screening system makes it possible to determine the activity, the chemoselectivity, the regioselectivity or the stereoselectivity, or all or some of these catalytic properties.

13. A catalyst which can be obtained by chemically linking a synthetic metal-containing or metal-free catalytically active center to an enzyme or protein mutant, in accordance with one of claims 1-12.
